(19) United States
(12) Patent Application Publication
(10) Pub. No.: US 2010/0036201 A1
(43) Pub. Date: Feb. 11, 2010

(54) FLEXIBLE TUBE FOR ENDOSCOPE AND ITS MANUFACTURING PROCESS

(76) Inventor: Hitoshi OGURA, Higashiyamato-shi (JP)

Correspondence Address: OSTROLENK FABER GERB & SOFFEN, 1180 AVENUE OF THE AMERICAS, NEW YORK, NY, 100368403, US

(21) Appl. No.: 12/424,821

(22) Filed: Apr. 16, 2009

(30) Foreign Application Priority Data
Aug. 7, 2008 (JP) 2008-204457

(51) Int. Cl. A61B 1/005; B05D 3/00

(52) U.S. Cl. 600/121; 427/2.1

(57) ABSTRACT

The present invention provides an endoscopic flexible tube, including a spiral tube, a mesh tube covering the spiral tube, and a sheath coating the outer surface of the mesh tube, the sheath being composed of a sheathing resin material containing closed cells.

### CROSS-REFERENCE TO RELATED APPLICATIONS

This application is based upon and claims the benefit of priority from prior Japanese Patent Application No. 2008-204457, filed Aug. 7, 2008, the entire contents of which are incorporated herein by reference.

### BACKGROUND OF THE INVENTION

1. Field of the Invention

The present invention relates to a flexible tube, and specifically to an endoscopic flexible tube and a method for producing the same.

2. Description of the Related Art

Endoscopes are reused, and must be washed and disinfected after every use. Therefore, the sheaths of endoscopic flexible tubes must be impermeable to body fluids, cleaning solvents, and antiseptic solutions, and must have elasticity and flexibility suitable for insertion into body cavities.

Conventionally, the sheath of an endoscopic flexible tube is composed of a mixture of a thermoplastic polyester elastomer (TPC) and a thermoplastic polyurethane elastomer (TPU), or a mixture of TPU and TPC containing soft polyvinyl chloride (PVC). Jpn. Pat. Appln. KOKAI Publication No. 11-56762 suggests a product composed of a thermoplastic fluorocarbon elastomer.

### BRIEF SUMMARY OF THE INVENTION

A first aspect of the present invention relates to an endoscopic flexible tube, including a spiral tube, a mesh tube covering the spiral tube, and a sheath coating the outer surface of the mesh tube, wherein the sheath is composed of a sheathing resin material containing closed cells.

A second aspect of the present invention relates to the endoscopic flexible tube, wherein the closed cells are preferably formed with hollow microspheres.

A third aspect of the present invention relates to the endoscopic flexible tube, wherein the hollow microspheres are preferably made of glass.

A fourth aspect of the present invention relates to the endoscopic flexible tube, wherein the hollow microspheres have an average particle size of 5 to 135 μm.

A fifth aspect of the present invention relates to the endoscopic flexible tube, wherein the loading of the hollow microspheres is 45 parts by weight or less with respect to 100 parts by weight of the sheathing resin material.

A sixth aspect of the present invention relates to a method for producing an endoscopic flexible tube, preferably including covering a spiral tube with a mesh tube to form a flexible tube core, kneading a sheathing resin material together with hollow microspheres to make a sheathing material, and coating the outer surface of the flexible tube core with the sheathing material to obtain an endoscopic flexible tube.

Advantages of the invention will be set forth in the description which follows, and in part will be obvious from the description, or may be learned by practice of the invention. Advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate embodiments of the invention, and together with the general description given above and the detailed description of the embodiments given below, serve to explain the principles of the invention.

FIG. 1 is a perspective view showing the schematic structure of an endoscope 10 according to a first embodiment;

FIG. 2 is a schematic view of an endoscopic flexible tube 1 according to the first embodiment;

FIG. 3 is a cross-sectional view of the endoscopic flexible tube 1 according to the first embodiment;

FIG. 4 is an enlarged view of a sheath 6 according to the first embodiment; and

FIG. 5 shows schematic views of the sheath 6 in various forms.

### DETAILED DESCRIPTION OF THE INVENTION

Hereinafter, embodiments of the invention will be described with reference to the drawings. Incidentally, the embodiments described below are merely examples for explaining the structure of the invention in detail. Accordingly, the invention should not be restrictively interpreted based on the description of the following embodiments. The scope of the invention includes all embodiments including various modifications and improvements of the following embodiments within the scope of the invention as set forth in the claims.

### First Embodiment

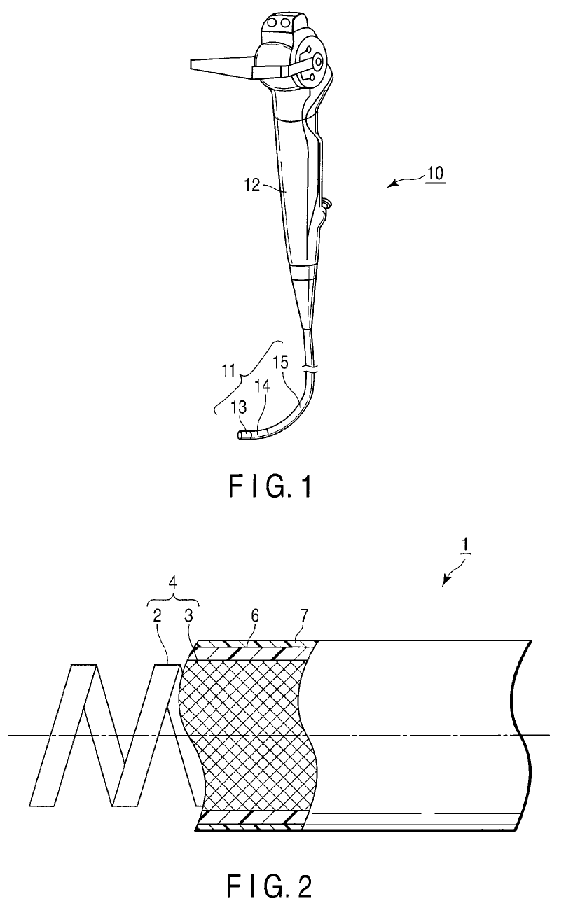

FIG. 1

FIG. 1 is a perspective view showing the schematic structure of an endoscope 10 according to a first embodiment. As shown in FIG. 1, the endoscope 10 includes an elongated flexible insert 11, and an operating unit 12 provided at the base of the insert 11. The insert 11 is composed of a toughened tip 13, a bending section 14 connected to the tip 13, and a flexible tube 15 connected to the bending section 14. The bending section 14 can be bent to a desired angle by remote control with the operating unit 12. The flexible tube 15 is a hose for inserting the tip 13 deeply into, for example, the body cavity such as the duodenum, small intestine, or large intestine.

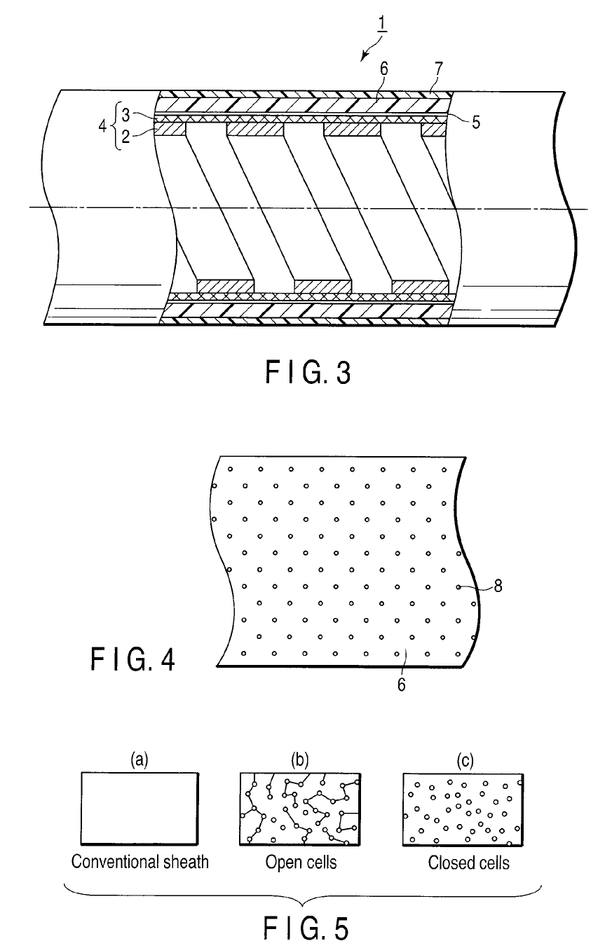

FIGS. 2 and 3

FIGS. 2 and 3 are a schematic view and a cross-sectional view of the endoscopic flexible tube 1 according to the first embodiment, respectively.

As shown in FIGS. 2 and 3, the endoscopic flexible tube (hereinafter referred to as “flexible tube”) 1 is composed of a flexible tube core 4, and a sheath 6 coating the outer surface of the flexible tube core 4. The flexible tube core 4 is composed of a spiral tube 2, and a mesh tube 3 coating the outer surface of the spiral tube 2.

The spiral tube 2 is a spirally wound elastic sheet. The elastic sheet may be made of stainless steel or a copper alloy. The mesh tube 3 is woven from a plurality of metallic or nonmetallic threads. The threads may be made of stainless steel or a synthetic resin. In order to improve the adhesion with the sheathing resin, the mesh tube 3 may be woven from a mixture of metallic and nonmetallic threads.

The sheath 6 coating the outer surface of the flexible tube core 4 may be composed of any known thermoplastic elastomer such as a thermoplastic polyester elastomer (TPC), a thermoplastic polyurethane elastomer (TPU), a thermoplastic fluorocarbon elastomer, a thermoplastic olefin elastomer, or a thermoplastic styrene elastomer. The sheath 6 may be composed of a crosslinked rubber.

The outer surface of the sheath 6 may be further coated with a top coat 7. The top coat 7 is made of a thin material with excellent chemical resistance and smoothness on the body wall of a patient. The top coat 7 may be made of a urethane-based or fluorocarbon resin. In the endoscopic flexible tube 1 of the present invention, the flexible tube core 4 is coated with a sheath containing closed cells. Therefore, the endoscopic flexible tube 1 having the sheath 6 as the outermost layer provides high elasticity and impermeability, regardless of the presence or absence of the top coat 7. Therefore, the step of forming the top coat 7 may be omitted from the manufacturing process of the endoscopic flexible tube 1, thereby achieving the scale-down of the manufacturing apparatus, labor savings in procurement and preparation of raw materials, and reduction of the manufacturing cost. In this case, the weight and diameter of the endoscopic flexible tube 1 are further reduced.

Alternatively, as shown in FIG. 3, a layer of a coupling agent 5 may be provided on the outer surface of the flexible tube core 4, the layer being closely coated with the sheath 6 composed of a thermoplastic elastomer. The coupling agent 5 may be a silane coupling agent. Other examples of the coupling agent include titanate-based, aluminum-based, and zirconium-based coupling agents. The coupling agent may contain a pigment for facilitating the distinction of whether the coupling agent is applied.

FIGS. 4 and 5

FIG. 4 is an enlarged view of the sheath 6 according to the first embodiment. The sheath 6 contains a plurality of closed cells 8. FIG. 5 shows schematic views of the sheath 6 in various forms.

As shown in FIG. 4, a plurality of closed cells 8 are dispersed in the sheath 6 of the flexible tube according to the present invention. The closed cells 8 dispersed in the sheath 6 reduce the specific gravity of the sheath 6, thereby achieving the high elasticity of the flexible tube 1.

FIG. 5 shows different sheath structures. The conventional sheath 6 is composed of a uniform resin material (FIG. 5A). The conventional sheath 6 has a high specific gravity because it contains no cavity, and thus cannot provide high elasticity. On the other hand, a sheath produced with, for example, a foaming agent contains open cells (FIG. 5B). The sheath 6 containing open cells is not completely impermeable to water. Therefore, the sheath 6 allows penetration of pathogens thereinto, which hinders sufficient sterilization. Even if cavities are formed using a foaming agent, the cavities disappear over time, which results in the thinning of the sheath and the variation of the outside diameter. On the other hand, the sheath 6 according to the first embodiment contains closed cells formed with hollow microspheres or a porous material (FIG. 5C). Since the sheath 6 containing closed cells has a low specific gravity, it provides high elasticity. In addition, since the cavities are independent, the sheath 6 has water impermeability equivalent to the conventional sheath, and does not allow penetration of pathogens thereinto. Therefore, the sheath 6 will not suffer from contamination, and thus is reusable after any treatment such as washing, disinfection, or low-temperature plasma sterilization. Since the cavities will not be disappeared by heating, the sheath 6 may be sterilized with high-pressure steam in an autoclave thereby more securely preventing infection, and the ease of insertion is maintained in the long term.

The closed cells contained in the sheath 6 according to the present invention may be formed through the addition of hollow microspheres. The hollow microspheres are fine hollow bodies having a cavity inside. The hollow microspheres have a high strength and high heat resistance, and will not be collapsed or melted during molding of the resin material composing the sheath 6. Therefore, the hollow microspheres provide stable closed cells within the sheath 6. The hollow microspheres may be made of, for example, glass or a resin.

The average particle size of the hollow microspheres is from 1 to 1000 μm, preferably from 2 to 500 μm, more preferably from 3 to 200 μm, and most preferably from 5 to 135 μm. Hollow microspheres having a smaller average particle size are more finely dispersed to increase the elongation of the sheathing resin material.

The density of the hollow microspheres depends on the density of the sheathing resin material. The specific gravity of the sheath 6 containing hollow microspheres must be lower than that composed of a resin material alone and containing no microsphere. In usual cases, the true density of the hollow microspheres is 1.8 g/cm³ or less, preferably 1.1 g/cm³ or less, the apparent density is 1.5 g/cm³ or less, preferably 0.8 g/cm³ or less, and the bulk density is 1.0 g/cm³ or less, preferably 0.5 g/cm³ or less. The porosity of the hollow microspheres is from 10 to 99%, preferably from 50 to 98%, and more preferably from 70% to 95%. Hollow microspheres having a smaller true density more reduce the specific gravity of the sheathing material, and are highly effective in a small dose.

The hollow microspheres are thermally stable, and specifically have a softening temperature of 300° C. or higher, preferably 500° C. or higher, and more preferably 550° C. or higher. The hollow microspheres are highly resistant to pressure, and specifically has a pressure capacity of 0.1 MPa or more, preferably 1.0 MPa or more, and more preferably 1.7 MPa or more.

The hollow glass microspheres are composed of, for example, SiO₂, B₂O₃, Na₂O, CaO, Al₂O₃, Fe₂O₃, K₂O, MgO, ZnO, TiO₂, or P₂O₅. The hollow glass microspheres may be made of soda lime borosilicate glass. High-quality commercial hollow glass microspheres are widely available, and examples thereof include GLASS BUBBLES (Sumitomo 3M Ltd.), Q-CELL (Potters Industries Inc.), and E-SPHERES (Taiheiyo Cement Corporation). The flexible tube 1 according to the present invention may be produced with these microspheres. Hollow resin microspheres are composed of, for example, a thermosetting resin such as an epoxy or phenol resin.

These hollow microspheres may be used alone or in combination.

Hollow microspheres are usually classified into fillers. If the loading of the hollow microspheres is too high, the sheathing material may be too hard or brittle. Accordingly, the loading of the hollow microspheres is 50 parts by weight or less, and particularly preferably 45 parts by weight or less with respect to 100 parts by weight of the sheathing resin material, in order to reduce the specific gravity of the sheath 6 to increase its elasticity while preventing embrittlement.

Alternatively, a porous material may be used in place of the hollow microspheres to obtain the same structure.

Other fillers may be added as necessary. Specific examples of the fillers include inorganic fillers such as carbon black, silica, barium sulfate, titanium oxide, aluminum oxide, calcium carbonate, calcium silicate, magnesium silicate, and aluminum silicate, and organic fillers such as polytetraluoroethylene resins, polyethylene resins, polypropylene resins, phenolic resins, polyimide resins, melamine resins, and silicone resins. These fillers may be used in combination.

In addition, fibers may be added as desired. Specific examples of the fibers include inorganic fibers such as glass fibers, alumina fibers, and rook wool, and organic fibers such as cotton, wool, silk, hemp, nylon fiber, aramid fibers, vinylon fibers, polyester fibers, rayon fibers, acetate fibers, phenol-formaldehyde fibers, polyphenylenesulfide fibers, acrylic fibers, polyvinyl chloride fibers, polyvinylidene chloride fibers, polyurethane fibers, and tetrafluoroethylene fibers. These fibers may be used in combination.

In addition, other additives such as lubricants, stabilizers, weathering stabilizers, ultraviolet absorbers, and antistatic agents may be added without impairing the advantages of the invention.

The method for producing the endoscopic flexible tube 1 according to the first embodiment will be described below.

First, a spiral tube 2 composed of a spirally wound elastic sheet, such as a stainless steel, is covered by a mesh tube 3 woven from, for example, synthetic resin threads.

Next, a layer of a coupling agent 5 is provided on the outer surface of the mesh tube 3, and the top of the layer is closely coated with a sheath 6. The layer of the coupling agent 5 is not essential, and the sheath 6 may be provided directly on the mesh tube 3. In this case, it is preferred that the mesh tube 3 is preliminarily coated with a highly penetrative adhesive such as a urethane adhesive.

The formation of the sheath 6 according to the present invention may use various conventional methods. Usually, melt kneading with, for example, a kneader, a Banbury mixer, or a continuous kneading extruder is used. The kneading temperature is not particularly limited, insofar as the components are uniformly dispersed, and the temperature is not higher than the decomposition temperature of the materials. Specifically, the sheath 6 is formed by mixing, for example, a thermoplastic polyester elastomer (TPC) with a specified amount of hollow glass microspheres, kneading the mixture with a continuous kneading extruder, and the kneaded product is extruded to form the sheath 6.

Finally, a resin with excellent chemical resistance and smoothness, such as a urethane or fluorocarbon resin, is deposited on the sheath 6 to a predetermined thickness by dipping or extrusion, thereby forming the top coat 7. The formation of the top coat 7 may be carried out concurrently with coating with the sheath 6. Since the sheath 6 according to the first embodiment contains closed cells, it has a low specific gravity and high elasticity, as well as excellent impermeability and chemical resistance. Therefore, the top coat 7 is not essential for the flexible tube 1 including the sheath 6 according to the first embodiment.

### EXAMPLE

The present invention will be further described with reference to the following examples, but the present invention is not limited to the examples. 1. Making of Endoscopic Flexible Tube

The elastomers and fillers (hollow glass microspheres) listed in Table 1 were used to make the sheaths 6, with which endoscopic flexible tubes were produced. The elastomers were polyester, polyurethane, polyolefin, and fluorocarbon elastomers, and the fillers were GLASS BUBBLES K1 and A20 (Sumitomo 3M Ltd.), Q-CELL 7014 (Potters Industries, Inc.), and E-SPHERES (Taiheiyo Cement Corporation).

TABLE 1
Recipe (unit: parts by weight)

| | | Examples | | | | | | Comparative Examples | | | |
|---|---|---|---|---|---|---|---|---|---|---|---|
| | | 1 | 2 | 3 | 4 | 5 | 6 | 1 | 2 | 3 | 4 |
| Elastomers | Polyester | 100 | 100 | — | — | — | 100 | 100 | 100 | 100 | — |
| | elastomer | | | | | | | | | | |
| | Polyurethane | — | — | 100 | — | — | — | — | — | — | — |
| | elastomer | | | | | | | | | | |
| | Polyolefin | — | — | — | 100 | — | — | — | — | — | — |
| | elastomer | | | | | | | | | | |
| | Fluorocarbon | — | — | — | — | 100 | — | — | — | — | 100 |
| | elastomer | | | | | | | | | | |
| Fillers | GLASS BUBBLES K1 | 10 | 40 | — | — | 40 | — | — | — | 80 | — |
| | true density | | | | | | | | | | |
| | 0.125 g/cm³ | | | | | | | | | | |
| | GLASS BUBBLES A20 | — | — | 20 | — | — | — | — | — | — | — |
| | true density | | | | | | | | | | |
| | 0.20 g/cm³ | | | | | | | | | | |
| | Q-CELL 7014 | — | — | — | 40 | — | — | — | — | — | — |
| | true density | | | | | | | | | | |
| | 0.14 g/cm³ | | | | | | | | | | |
| | E-SPHERES | — | — | — | — | — | 20 | — | — | — | — |
| | true density | | | | | | | | | | |
| | 0.7 to 0.8 g/cm³ | | | | | | | | | | |

| | | | | | | | | | | |
|---|---|---|---|---|---|---|---|---|---|---|
| Foaming agent | — | — | — | — | — | — | — | 0.1 | — | — |

### Example 1

10 parts by weight of hollow glass microspheres (GLASS BUBBLES K1, true density: 0.125 g/cm³) were mixed with 100 parts by weight of a thermoplastic polyester elastomer (TPC), and the mixture was kneaded with a continuous kneading extruder to obtain a sheathing material. A core composed of a spiral tube covered by a mesh tube was coated on its outer surface with the sheathing material, thereby producing an endoscopic flexible tube.

### Example 2

40 parts by weight of hollow glass microspheres (GLASS BUBBLES K1, true density: 0.125 g/cm³) were mixed with 100 parts by weight of a thermoplastic polyester elastomer (TPC), and the mixture was kneaded with a continuous kneading extruder to obtain a sheathing material. A core composed of a spiral tube covered by a mesh tube was coated on its outer surface with the sheathing material, thereby producing an endoscopic flexible tube.

### Example 3

20 parts by weight of hollow glass microspheres (GLASS BUBBLES A20, true density: 0.20 g/cm³) were mixed with 100 parts by weight of a thermoplastic polyurethane elastomer (TPU), and the mixture was kneaded with a continuous kneading extruder to obtain a sheathing material. A core composed of a spiral tube covered by a mesh tube was coated on its outer surface with the sheathing material, thereby producing an endoscopic flexible tube.

### Example 4

40 parts by weight of hollow glass microspheres (Q-CELL 7014, true density: 0.14 g/cm³) were mixed with 100 parts by weight of a thermoplastic polyolefine elastomer, and the mixture was kneaded with a continuous kneading extruder to obtain a sheathing material. A core composed of a spiral tube covered by a mesh tube was coated on its outer surface with the sheathing material, thereby producing an endoscopic flexible tube.

### Example 5

40 parts by weight of hollow glass microspheres (GLASS BUBBLES K1, true density: 0.125 g/cm³) were mixed with 100 parts by weight of a thermoplastic fluorocarbon elastomer, and the mixture was kneaded with a continuous kneading extruder to obtain a sheathing material. A core composed of a spiral tube covered by a mesh tube was coated on its outer surface with the sheathing material, thereby producing an endoscopic flexible tube.

### Example 6

20 parts by weight of hollow glass microspheres (E-SPHERES, true density: 0.7 to 0.8 g/cm³) were mixed with 100 parts by weight of a thermoplastic polyester elastomer (TPC), and the mixture was kneaded with a continuous kneading extruder to obtain a sheathing material. A core composed of a spiral tube covered by a mesh tube was coated on its outer surface with the sheathing material, thereby producing an endoscopic flexible tube.

### Comparative Example 1

A core composed of a spiral tube covered by a mesh tube was coated on its outer surface with a sheathing material composed of a thermoplastic polyester elastomer (TPC) alone and containing no hollow glass microsphere, thereby producing an endoscopic flexible tube.

### Comparative Example 2

0.1 part by weight of a foaming agent (VINYFOR AC #3, Eiwa Chemical Ind. Co., Ltd.) was added to 100 parts by weight of a thermoplastic polyester elastomer (TPC) containing no hollow glass microsphere, thus obtaining a sheathing material containing open cells. A core composed of a spiral tube covered by a mesh tube was coated on its outer surface with the sheathing material, thereby producing an endoscopic flexible tube.

### Comparative Example 3

In order to examine the influence of an excess amount of hollow glass microspheres, 80 parts by weight of hollow glass microspheres (GLASS BUBBLES K1, true density: 0.125 g/cm³) were mixed with 100 parts by weight of a thermoplastic polyester elastomer, and the mixture was kneaded with a continuous kneading extruder to obtain a sheathing material. A core composed of a spiral tube covered by a mesh tube was coated on its outer surface with the sheathing material, thereby producing an endoscopic flexible tube.

### Comparative Example 4

In order to examine the influence of an elastomer with a high specific gravity, a core composed of a spiral tube covered by a mesh tube was coated on its outer surface with a sheathing material composed of a thermoplastic fluorocarbon elastomer alone and containing no hollow glass microsphere, thereby producing an endoscopic flexible tube.

2. Test Result

TABLE 2

| | | Water | |
|---|---|---|---|
| | Elasticity | impermeability | Flexibility |
| Example 1 | ⊚ | ◯ | ◯ |
| Example 2 | ⊚ | ◯ | ◯ |
| Example 3 | ⊚ | ◯ | ◯ |
| Example 4 | ⊚ | ◯ | ◯ |
| Example 5 | ⊚ | ◯ | ◯ |
| Example 6 | ◯ | ◯ | ◯ |
| Comparative | Δ | ◯ | ◯ |
| Example 1 | | | |
| Comparative | ⊚ | X | ◯ |
| Example 2 | | | |
| Comparative | ⊚ | ◯ | X |
| Example 3 | | | |
| Comparative | X | ◯ | ◯ |
| Example 4 | | | |

The symbols represent the followings.
⊚: Markedly good,
◯: Good,
Δ: Acceptable,
X: Unacceptable

As is evident from Table 2, the flexible tubes produced in Examples 1 to 6 received good ratings for the elasticity, water impermeability, and flexibility. On the other hand, the flexible tubes produced in Comparative Examples 1 to 4 received an unsatisfactory rating for the elasticity, water impermeability, or flexibility.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An endoscopic flexible tube comprising:
a spiral tube;
a mesh tube which covers the spiral tube; and
a sheath which coats an outer surface of the mesh tube,
wherein the sheath is composed of a sheathing resin material containing closed cells.

2. The endoscopic flexible tube according to claim 1, wherein the closed cells are formed with hollow microspheres.

3. The endoscopic flexible tube according to claim 2, wherein the hollow microspheres are made of glass.

4. The endoscopic flexible tube according to claim 2, wherein the hollow microspheres have an average particle size of 5 to 135 μm.

5. The endoscopic flexible tube according to claim 2, wherein loading of the hollow microspheres is 45 parts by weight or less with respect to 100 parts by weight of the sheathing resin material.

6. A method for producing an endoscopic flexible tube, comprising:
covering a spiral tube with a mesh tube to form a flexible tube core;
kneading a sheathing resin material together with hollow microspheres to make a sheathing material; and
coating an outer surface of the flexible tube core with the sheathing material to obtain an endoscopic flexible tube.

7. The method for producing an endoscopic flexible tube according to claim 6, wherein loading of the hollow microspheres is 45 parts by weight or less with respect to 100 parts by weight of the sheathing resin material.
